(19) United States
(12) Plant Patent Application Publication
(10) Pub. No.: US 2003/0150039 P1
(43) Pub. Date: Aug. 7, 2003

(54) Verbena plant named 'Sunvivare'

(76) Inventors: Yasuyuki Murakami, Gamo-gun (JP); Yasunori Yomo, Yasu-gun (JP)

Correspondence Address: BURNS DOANE SWECKER & MATHIS L L P, POST OFFICE BOX 1404, ALEXANDRIA, VA, 22313-1404, US

(21) Appl. No.: 10/066,736

(22) Filed: Feb. 6, 2002

(51) Int. Cl. A01H 5/00

(52) U.S. Cl. PLT/308

(57) ABSTRACT

Disclosed herein is a new and distinct variety of Verbena plant having a semi-erect growth habit with medium stems and a very broad plant spreading area. The Verbena plant has abundant branching particularly with respect to primary branching, and great profusion of blooms with the entire plant remaining in bloom for a considerable period of time. The flowers are large with petals having a deep red color without eye color, and have a plentiful number of flowers in a spike. The blooming period is from late April to November and the flowering duration is long. The plant exhibits high tolerance to cold, moderate tolerance to heat, high resistance to pests and diseases, particularly powdery mildew, and high resistance to rain.

### BACKGROUND OF THE VARIETY

The present invention relates to a new and distinct variety of verbena plant obtained as a mutation of verbena plant variety Verbena hybrida ‘SUNVIVARO’ (Registration No. 8142 under the Seeds and Seedlings Law of Japan).

The verbena is a very popular plant and is used for flower bedding and potting in the summer season. There are only a few varieties of the verbena plant which have much branching, many flowers in a spike, a long flowering duration and a high resistance to cold, rain, diseases and pests. Accordingly, this invention is aimed at obtaining a new variety having much branching, many flowers in a spike, a long flowering duration and a high resistance to cold, rain, diseases and pests, and moderate resistance to heat, and also having a deep red color petal.

The new variety of verbena plant according to this invention originated as a mutation from Verbena hybrida ‘SUNVIVARO’, which was previously filed in Japan (Registration No. 8142 under the Seeds and Seedlings Law of Japan) and the United States of America (Application No. 375362 as a plant patent application).

The new variety Verbena hybrida ‘SUNVIVARE’ was discovered as a spontaneous branch mutation of the Verbena hybrida ‘SUNVIVARO’ in August 1998 in a field in Germany. The mutant verbena plant was propagated by cutting and was grown in pot and bedding trials from May 1999 in Yamanashi and Shiga prefectures, Japan. The botanical characteristics of the plant were examined, using a similar variety ‘SUNVIVARO’ (Registration No. 8142 under the Seeds and Seedlings Law of Japan) and ‘SUNVIVAPA’ (Registration No. 7395 under the Seeds and Seedlings Law of Japan) for comparison. As a result, it was concluded that this verbena is distinguishable from other varieties, whose existence is known, uniform and stable in its characteristics, and this new variety of verbena plant was named ‘SUNVIVARE’.

In the following description, the color-coding is in accordance with the Inter-Society Color Council—National Bureau of Standards (ISCC-NBS), the Horticultural Color Chart of The Royal Horticultural Society, London, England (R.H.S. Color Chart), and a color chart based on The Japan Color Standard for Horticultural Plant (J.H.S. Color Chart) is also added for reference.

The present variety ‘SUNVIVARE’ is a mutant of the ‘SUNVIVARO’ obtained from crossing a wild type of verbena plant native to south Brazil and Verbena peruviana. The ‘SUNVIVARO’ is our bred variety and was registered in Japan in 2000 (Registration No. 8142 under the Seeds and Seedlings Law of Japan) and was filed in the US in 1999 (Application No. 375362 as a plant patent application), and so on. The main botanical characteristics of the parent variety ‘SUNVIVARO’ are as follows.

Plant:

Growth habit.—Erect.

Extension.—Medium.

Height.—Medium to high.

Stem:

Diameter.—Medium; 1.4-3.0 mm.

Anthocyanin pigmentation.—Absent.

Pubescence.—Medium.

Prickle.—Absent.

Branching.—Medium.

Subterranean stem.—Absent.

Length of internode.—Short; 1.0-1.8 cm.

Leaf:

Phyllotaxis.—Opposite.

Shape of blade.—Hastate.

Blade incision.—Present.

Depth of blade incision.—Shallow.

Blade crenation of margin.—Serrate.

Length.—Medium; 3.8 cm.

Width.—Medium; 2.5 cm.

Color.—Grayish olive green. (R.H.S. Color Chart No. 137A, JHS Color Chart No. 3716)

Pubescence.—Sparse.

Petiole.—Present.

Diameter of petiole.—Medium; 1.8 mm.

Length of petiole.—Short; 2.6 mm.

Flower:

Shape of cluster.—Funnel-shaped.

Length of cluster.—Medium; 31 mm.

Diameter of cluster.—Medium to large; 45-54 mm.

Facing direction.—Upward.

Outward curvature of petal.—Slightly curved.

Diameter.—Large; 1.8-2.2 cm.

Height.—Medium.

Color.—Vivid purplish red. (R.H.S. Color Chart No. 66A, JHS Color Chart No. 9707).

Eye color.—Absent.

Variegation on petal.—Absent.

Color intensity.—Substantially even.

Overlapping of petals.—Separate.

Incision of petal.—Present.

Number of petals.—Medium; 4.

Incision of calyx.—Present.

Length of calyx.—Long; 1.2 mm.

Anthocyanin pigmentation of calyx limb.—Absent.

Shape of pistil.—Two lobes.

Number of stamens.—Medium.

Color of anther.—Yellowish green.

Diameter of peduncle.—Thin; 1.2 mm.

Length of Peduncle.—Long; 1.3 cm.

Number of flowers.—Many.

Reproductive organs.—1 pistil and 4 stamens.

Flower fragrance.—Absent.

Flowering time.—Medium.

Flowering duration.—Long.

Physiological and ecological characteristics: High resistance to diseases and pests, particularly powdery mildew. High tolerance to cold and rain. Moderate tolerance to heat.

The breed ‘SUNVIVAPA’ used as a comparison for the new variety ‘SUNVIVARE’ is one of our varieties and was registered in Japan in 1999 (Registration No. 7395 under the Seeds and Seedlings Law of Japan) and was applied for in US in 1999 (Application No. 375408 as a plant patent application) and so on. The main botanical characteristics of ‘SUNVIVAPA’ are as follows.

Plant:

Growth habit.—Erect.

Plant extension.—Medium; 60 cm.

Plant height.—High. (40-50 cm).

Stem:

Diameter.—Medium. (2-3 mm).

Anthocyanin pigmentation.—Present.

Pubescence.—Dense.

Prickle.—Absent.

Branching.—Abundant.

Subterranean stem.—Absent.

Length of internode.—Medium. (4-5 cm).

Leaf:

Phyllotaxis.—Opposite.

Shape of blade.—Hastate.

Blade of incision.—Present.

Depth of blade incision.—Shallow.

Crenation of blade margin.—Crenated.

Length.—Medium. (4-5 cm).

Width.—Medium. (3 cm).

Color.—Moderate olive green. (R.H.S. Color Chart No. 146A, JHS Color Chart No. 3509).

Pubescence.—Medium.

Petiole.—Present.

Petiole diameter.—Medium; 1.5 mm.

Petiole length.—Medium; 5.0 mm.

Flower:

Shape of cluster.—Funnel-shaped.

Cluster length.—Medium. (3 cm).

Cluster diameter.—Large. (4-5 cm).

Facing direction.—Upward.

Outward curvature of petal.—Slightly curved.

Diameter.—Large. (20-30 mm).

Height.—Medium; 1.5 cm.

Color of petal.—Vivid reddish purple (R.H.S. Color Chart No. 78A, JHS Color Chart No. 8907).

Eye color.—Absent.

Variegation.—Absent.

Color intensity.—Absent.

Overlapping of petals.—Separate.

Incision of petal.—Present.

Number of petal.—Medium; 5.

Calyx incision.—Present.

Calyx length.—Medium; 1.3 cm.

Anthocyanin pigmentation of calyx limb.—Present.

Shape of pistil.—Two lobes.

Number of stamens.—Medium.

Color of anther.—Yellowish green.

Peduncle diameter.—Medium; 2.0 cm.

Peduncle length.—Medium; 2.0 cm.

Number of flowers.—Medium.

Reproductive organs.—1 pistil and 4 stamens.

Flower fragrance.—Absent.

Flowering time.—Late.

Flowering duration.—Long.

Physiological and ecological characteristics:

Tolerance to cold.—High.

Tolerance to heat.—High.

Resistance to diseases.—High.

Resistance to pests.—High.

This new variety of verbena plant ‘SUNVIVARE’ was asexually reproduced by cutting at Youkaichi-shi, Shiga-ken, Japan and Kitakoma-gun, Yamanashi-ken, Japan, and the homogeneity and stability thereof were confirmed.

### SUMMARY OF THE VARIETY

This new variety of verbena plant has a semi-erect habit and plant height is high. The plant has much branching and a plentiful number of flowers in a spike, and great profusion of blooms. The Blooming period is from late April to November, and flowers are still open in the rainy season and flowering duration is long. The whole bush remains in bloom for a considerable period of time. The flower size is large and the petal color of the flowers is deep red without eye color. The plant is highly tolerant to cold, moderately tolerant to heat, and has high resistance to pests and diseases, particularly powdery mildew, and high resistance to rain.

### BRIEF DESCRIPTION OF THE DRAWINGS

FIG. 1 is a photograph showing a partial view of the new variety of verbena plant planted in the ground;

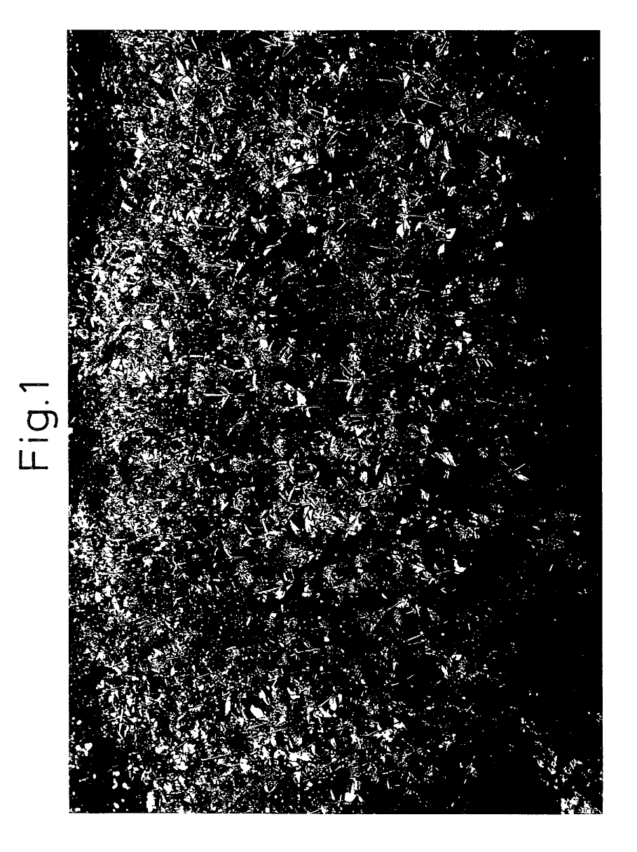

FIG. 2 is a photograph of flowers of the new variety of verbena plant.

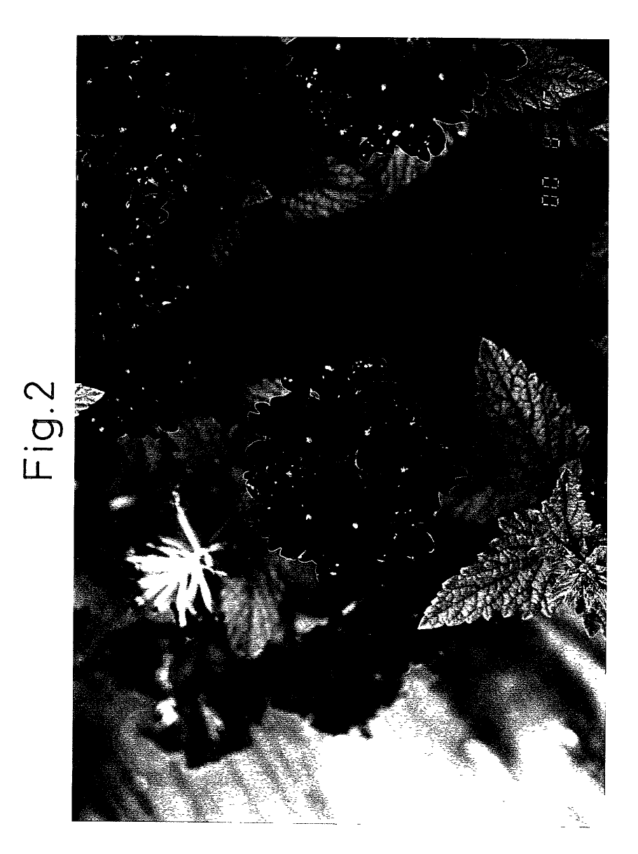

FIG. 3 is a photograph of a general view of the new variety of verbena plant planted in the ground.

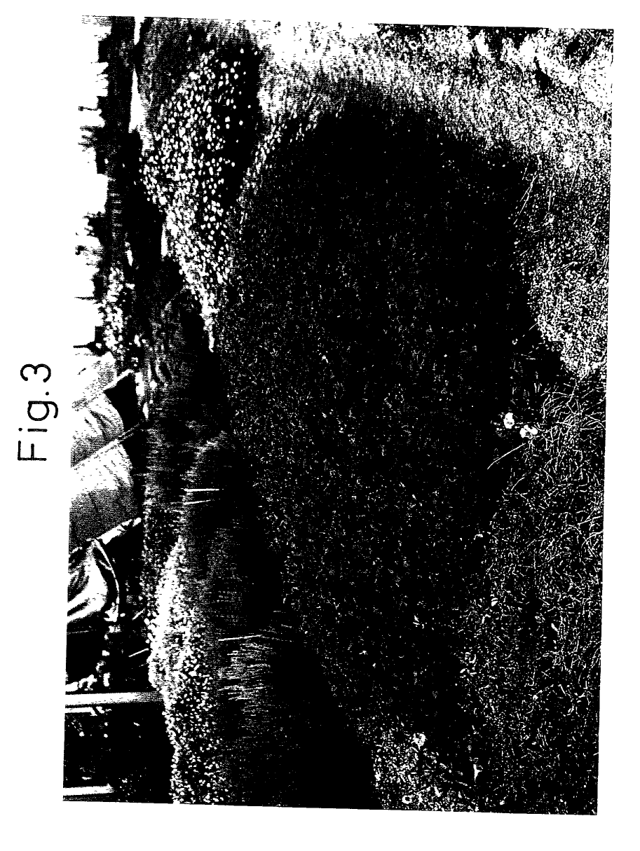

### DESCRIPTION OF THE NEW VARIETY

The botanical characteristics of the new and distinct variety of verbena plant named ‘SUNVIVARE’ are as follows, having observed, during October at Youkaichi-shi, Shiga-ken, Japan, young plants that had been reproduced by cutting during the preceding April.

Plant:

Growth habit.—Semi-erect.

Plant extension.—Broad. (70 cm).

Plant height.—High. (30 cm).

Stem:

Diameter.—Medium. (2.8 mm).

Anthocyanin pigmentation.—Absent.

Pubescence.—Medium to dense.

Prickle.—Absent.

Branching.—Medium.

Subterranean stem.—Absent.

Length of internode.—Medium (3.6 cm).

Leaf:

Phyllotaxis.—Opposite.

Shape of blade.—Hastate.

Blade of incision.—Present.

Depth of blade incision.—Shallow.

Crenation of blade margin.—Serrate.

Length.—Medium. (4.1 cm).

Width.—Medium. (2.9 cm).

Color.—Moderate olive green. (R.H.S. Color Chart No. 137B, J.H.S. Color Chart No. 3508).

Pubescence.—Medium.

Petiole.—Present.

Petiole diameter.—Medium. (1.3 mm).

Petiole length.—Short. (1.8 mm).

Flower:

Shape of cluster.—Funnel-shaped.

Cluster length.—Medium. (30 mm).

Cluster diameter.—Medium. (50 mm).

Facing direction.—Upward.

Outward curvature of petal.—Slightly curved.

Diameter.—Large. (18 mm).

Height.—Low. (0.5 cm).

Color of petal.—Deep red. (R.H.S. Color Chart No. 60A, J.H.S. Color Chart No. 0408).

Eye color.—Absent.

Variegation.—Absent.

Color intensity.—Absent.

Overlapping of petals.—Separate.

Incision of petal.—Present.

Number of petal.—Medium. (5).

Calyx incision.—Present.

Calyx length.—Long. (1.0 cm).

Anthocyanin pigmentation of calyx limb.—Absent.

Pistil shape.—Two lobes.

Stamen number.—Medium.

Anther color.—Yellow green.

Peduncle diameter.—Thin. (1.6 mm).

Peduncle length.—Short. (3.4 cm).

Number of flowers.—Abundant.

Reproductive organs.—1 pistil and 5 stamens.

Flower fragrance.—Absent.

Flowering time.—Medium.

Flowering duration.—Long.

Physiological and ecological characteristics:

Tolerance to cold.—High.

Tolerance to heat.—Medium.

Resistance to diseases.—High.

Resistance to pests.—High.

This new variety of Verbena plant is most suitable for flower bedding and potting, particularly in hanging pots and planters, and is further excellent for use as ground cover. Pinching of old blossoms will enhance the formation of new blossoms.

The plant of this new variety, ‘SUNVIVARE’ is presently planted and maintained at Youkaichi-shi, Shiga-ken, Japan.

We claim:

1. A new and distinct variety of Verbena plant, substantially as herein illustrated and described, characterized particularly as to novelty by a semi-erect growth habit, (B) a plentiful number of flowers in a spike and great profusion of blooms with the entire plant remaining in bloom for a considerable period of time, (C) along flowering duration, (D) the flowers are large and the petals have a deep red color without eye color, (E) a high resistance to rain, cold, drought, diseases and pests, and moderate resistance to heat and (F) an early flowering time.
